(19) United States
(12) Patent Application Publication
(10) Pub. No.: US 2026/0165922 A1
(43) Pub. Date: Jun. 18, 2026

(54) COMPOSITIONS AND METHODS FOR LIGHTENING HAIR

(71) Applicant: L'Oreal, Paris (FR)

(72) Inventors: Tathagata MUKHERJEE, Union, NJ (US); Martin ASARE, Springfield, NJ (US); Joshua LEWIS, Clark, NJ (US)

(21) Appl. No.: 18/986,307

(22) Filed: Dec. 18, 2024

(51) Int. Cl. A61K 8/23; A61K 8/25; A61K 8/365; A61K 8/44; A61Q 5/08

(52) U.S. Cl. CPC A61K 8/23; A61K 8/25; A61K 8/365; A61K 8/44; A61Q 5/08; A61K 2800/5922; A61K 2800/882

(57) ABSTRACT

The disclosure relates to compositions and methods for lightening the color of hair, wherein the compositions comprise one or more benefit agents that protect the hair from damage and/or reduce damage to the hair during a process for lightening the color of the hair. The compositions and methods provide very high levels of lift while minimizing damage to the hair.

### TECHNICAL FIELD

The present disclosure relates to compositions for lightening hair, kits comprising the compositions, and methods for using the compositions.

### BACKGROUND

It is known that consumers desire to use cosmetic and care compositions that enhance the appearance of keratinous substrates such as hair, e.g., by changing the color of the hair, and/or by imparting various properties to hair, such as shine and conditioning. The process of altering the color of hair may, for example, involve lifting the color of the hair to lighten the hair color, and/or depositing an artificial color onto the hair which provides a different or darker shade or color to the hair. The process of lifting the color of hair, also known as lightening, generally requires the use of compositions that comprise oxidizing agents. Such oxidizing agents enter the hair shaft and react with melanin in the hair, thereby lightening the color of the hair.

Variation of tone height before and after the application of a lightening composition is used to evaluate lightening or lifting of the color of the hair. Degree or level of lightening or lift corresponds to the variation. The notion of “tone” is based on the classification of the natural shades, one tone separating each shade from the shade immediately following or preceding it, which is well known to hairstyling professionals. Tone heights or levels range from 1 (black) to 10 (light blonde) or even up to 12 (platinum), one unit corresponding to one tone. Thus, the higher the number, the lighter the shade or the greater the degree of lift.

Many consumers seek high degrees of lift, such as when going from black to light blonde. The level of lift desired is typically achieved by using varying amounts of oxidizing and/or alkaline agents in the lightening composition, or by varying the length of time the composition is left on the hair. In cases where very high levels of lift are sought (e.g. at least 6), multiple applications of the lightening composition are often required to achieve the desired shade.

Additionally, consumers also often seek tones that are lighter and brighter. This can be achieved by altering the undertones of the hair color. Typically, undertones are altered or neutralized by applying a separate toning composition to the hair after the hair is lightened, for example to make the tone appear warmer or cooler, get rid of brassy undertones, etc.

However, compositions for lightening and toning hair are known to be harsh and to damage the hair fibers and/or irritate the scalp during the lightening treatment. Compositions and processes that are used for high (e.g. at least 6) levels of lift are particularly damaging due to the need for increased amounts of harsh chemicals to achieve the desired lightening effect and for increased processing times, meaning that the damaging compositions are in contact with the hair fibers for a longer period of time. In addition, sometimes it is necessary to perform the lightening process more than once, especially when high or very high levels of lift are desired. Such multiple processing applications are also particularly damaging to hair. All of these treatments therefore lead to hair that feels rough, tangles easily, and looks unhealthy. Thus, in order to reduce or avoid damage to the hair, as well as to improve the cosmetic performance of these compositions, new lightening compositions and/or processes for achieving the desired level of lift, preferably in less time, with fewer applications, and/or with reduced amounts of harsh chemicals, are needed.

Lightening compositions typically comprise persulfates which accelerate the speed of the hair lightening process. On the one hand this is advantageous because the harsh chemical treatment can be removed from the hair after a relatively shorter period of time, thus permitting less exposure of the hair fibers to the damaging compositions. On the other hand, however, persulfates themselves are a contributing cause of hair damage. Moreover, the reduced processing time must also be balanced against the amount of time needed to achieve the desired level of lift, because if the composition is removed too soon, the active agents will not have had sufficient time to de-colorize the hair fibers and the desired level of lightening will not be achieved.

There have been various attempts to mitigate hair damage during the lightening process. For example, some bleaching compositions contain additional components intended to reduce the damage to the hair fiber. However, this approach poses difficulties insofar as some components cause the compositions to be unstable, are detrimental to cosmetic attributes of the treated hair, and/or present other problems such as lack ease and uniformity of application, poor rheology or viscosity of the compositions, and/or reduced efficacy in lifting or lightening the color of the hair. Other compositions have been modified to remove certain damaging components, but these compositions are not able to achieve the desired level of lightening in the time the compositions can be left on the hair before damage occurs. It would, therefore, be desirable to provide compositions and methods that can lift the color of hair in an effective and expeditious manner, while leaving the hair with satisfactory cosmetic properties.

It has now been surprisingly discovered that hair lightening compositions according to the disclosure having reduced amounts of certain harsh chemicals such as persulfate compounds and oxidizing agents can be prepared which permit effective and efficient lightening, while also providing a smooth feel and healthy appearance of the treated hair.

### SUMMARY

The present disclosure relates to compositions and methods for lightening or lifting the color of the hair, which are able to achieve very high or even ultra-high levels of lift, even with lower amounts of persulfates and oxidizing agents. Damage during the lightening/bleaching process is minimized, which protects the integrity of the hair and leaves the hair stronger, softer, smoother, and with a healthier appearance than hair lightened with traditional hair bleaching compositions.

The disclosure relates to base lightening compositions which comprise reduced amounts of harsh chemicals such as persulfate compounds. The base lightening compositions comprise (i) at least one persulfate compound, for example at least two persulfate compounds, (ii) at least one silicate compound, (iii) at least one benefit agent, and (iv) at least one carboxylic acid or salt.

The total amount of (i) persulfate compounds may vary, but is advantageously less than about 50%, such as less than about 45%, or less than about 40%, for example may range from about 10% to not greater than 50% or from about 25% to about 45% by weight, relative to the total weight of the base lightening composition.

In some embodiments, the base lightening compositions comprise (i) (a) ammonium persulfate and (b) at least one additional persulfate compound such as potassium persulfate and/or sodium persulfate. It has been discovered that choosing amounts of persulfate compounds relative to each other, very high levels of lift can be achieved in the same or even less time than with traditional base lightening compositions, leading to reduced damage to the hair. For example, in various embodiments the base lightening compositions may comprise ammonium persulfate and at least one additional persulfate compound, where the weight ratio of the amount of (a) ammonium persulfate to the total amount of (b) additional persulfate compounds is equal to or less than about 1, for example ranges from about 0.1 to about 0.9, such as from about 0.2 to about 0.8, from about 0.3 to about 0.7, or from about 0.4 to about 0.6.

The total amount of (ii) silicate compounds may vary, but generally ranges from about 5% to about 50%, such as from about 15% to about 45%, or from about 25% to about 40% by weight, based on the total weight of the base lightening composition. Optionally the base lightening compositions comprise at least one metasilicate, for example sodium metasilicate. In some embodiments, the base lightening compositions comprise at least two silicate compounds where at least one is a metasilicate, for example sodium silicate and sodium metasilicate.

Benefit agents surprisingly provide strength and fiber integrity benefits to the treated hair. The total amount of benefit agents can vary, but generally ranges from about 0.5% to about 8%, such as from about 0.75% to about 6%, from about 1% to about 5%, from about 1.5% to about 4.5%, from about 2% to about 4%, or from about 2.5% to about 3.5% by weight, relative to the total weight of the base lightening composition.

Carboxylic acids that can be used include mono, di, and tricarboxylic acids. Preferably, salts of carboxylic acids are used. The total amount of carboxylic acids and salts can vary, but generally ranges from about 0.5% to about 8%, such as from about 1% to about 6%, from about 1.5% to about 5.5%, from about 2% to about 5%, from about 2.5% to about 4.5%, or from about 3% to about 4% by weight, relative to the total weight of the base lightening composition.

The base lightening compositions may further comprise additional components such as alkalizing agents, thermal control agents, oxidizing agents, oils, fillers, thickening agents, anionic surfactants, and auxiliary components such as preservatives, hair conditioning agents, vitamins, chelating agents, pigments or colorants to color the composition, or any other component typically used in hair color lightening base compositions. Useful alkalizing agents include both organic and mineral alkalizing agents. Useful thermal control agents may include urea, sodium stearate, and/or mineral oil. Thermal control agents that can be used may, in various embodiments, permit the hair lightening composition to achieve a peak temperature that is less than 70° C., for example less than about 65° C., less than about 63° C., less than about 60° C., less than about 58° C., less than about 55° C., less than about 53° C., or less than about 50° C.

In other embodiments the disclosure relates to base lightening mixtures, which are formed by mixing or combining a base lightening composition according to the disclosure with a solvent, for example water. By mixing or combining the base lightening composition with a solvent prior to or at the same time as mixing the base composition with an oxidizing agent, the risk of damage to the hair or scalp is reduced. In various embodiments, the amount of solvent that can be mixed with or added to the base lightening composition may range from about 1% to about 25%, such as from about 5% to about 20% by weight, relative to the total weight of the base lightening composition. In some embodiments, the solvent is added to the base lightening composition, or mixed together with the base composition and oxidizing agent, so that a weight ratio of total persulfates in the base lightening composition to the amount of solvent added ranges from about 1:1 to about 5:1, such as from about 1:1 to about 4:1, or from about 1.5:1 to about 3.5:1. In still other embodiments, the amount of solvent that can be mixed with or added to the base lightening composition may be chosen to provide a weight ratio of the total weight of the base lightening composition to the total weight of solvent ranging from about 5:1 to about 15:1, such as from about 6:1 to about 14:1, or about 7:1 to about 13.5:1.

The disclosure further relates to methods of preparing hair lightening compositions comprising mixing or combining (a) a base lightening composition, (b) solvent, and (c) oxidizing agent, as well as to hair lightening compositions prepared accordingly. Optionally, the (a) base lightening composition and (b) solvent may first be mixed to form a base lightening mixture, and subsequently the base lightening mixture and (c) oxidizing agent may be mixed to form a hair lightening composition according to the disclosure. For example, in various embodiments the mix ratio of [base lightening mixture]:[oxidizing composition] can range from about 1:3 to about 3:1, such as from about 1:2.5 to about 2.5:1, from about 1:2 to about 2:1, or from about 1:1.5 to about 1.5:1, or may be about 1:1. In other embodiments, however, the (a) base lightening composition, (b) solvent, and (c) oxidizing agent may all be mixed together at approximately the same time.

In other embodiments, instead of combining the base lightening composition with a solvent, an additional amount of an aqueous oxidizing composition can be used to form a hair lightening composition. For example, rather than adding from about 1% to about 25% of a solvent to the base composition, the amount of aqueous oxidizing composition mixed with the base lightening composition can be increased by about 1% to about 25%, all weights relative to the total weight of the base lightening composition. By way of example, the additional amount of aqueous oxidizing composition may be chosen so that a weight ratio of total persulfates in the base lightening composition to the additional amount of aqueous oxidizing composition ranges from about 1:1 to about 5:1, such as from about 1:1 to about 4:1, or from about 1.5:1 to about 3.5:1. In still other embodiments, the additional amount of aqueous oxidizing composition can be chosen to provide a weight ratio of the total weight of the base lightening composition to the total weight of additional aqueous oxidizing composition ranging from about 5:1 to about 15:1, such as from about 6:1 to about 14:1, or about 7:1 to about 13.5:1. The disclosure thus further relates to methods of preparing hair lightening compositions comprising mixing or combining (a) a base lightening composition, and (b) an oxidizing composition comprising an additional amount of aqueous oxidizing composition, as well as to hair lightening compositions prepared accordingly.

In further embodiments, a combination of solvent and aqueous oxidizing composition can be used.

In still further embodiments, a base lightening composition according to the disclosure is mixed with an oxidizing composition without the addition of solvent or additional oxidizing composition.

The disclosure also relates to methods of lightening the color of hair using hair lightening compositions according to the disclosure, in particular method for achieving very high or even ultra-high levels of lift.

In further embodiments still, the disclosure relates to kits comprising at least two, for example at least three, compartments or containers, wherein a first compartment or container comprises a base lightening composition according to the disclosure. Additional compartment(s) or container(s) may, for example, comprise an oxidizing composition or oxidizing agent, a solvent for mixing with the base lightening composition to prepare the base lightening mixture, an implement to mix the base composition and an oxidizing composition or oxidizing agent and/or to apply the mixture to the hair, or one or more additional hair care compositions, for example comprising a pre- and/or post-treatment hair conditioning composition.

### BRIEF DESCRIPTION OF THE FIGURES

FIGS. 1A-1B are graphs showing the ΔE values of hair treated with comparative hair lightening compositions and hair lightening compositions according to the disclosure at T₁ and T₂.

FIGS. 1C-1D are plots showing the ΔL and Δb values of hair treated with comparative hair lightening compositions and hair lightening compositions according to the disclosure at T₁.

FIGS. 1E-1F are plots showing the ΔL and Δb values of hair treated with comparative hair lightening compositions and hair lightening compositions according to the disclosure at T₂.

FIG. 2 is a plot showing break stress and elastic modulus of swatches S1-S8, S11, S18, and S22 at T₂.

### DETAILED DESCRIPTION

The disclosure relates to compositions that are useful for lightening or lifting the color of the hair, methods of lightening or lifting the color of the hair using the compositions, and kits that can be used in the methods.

### I. Compositions

It has been surprisingly and unexpectedly discovered that hair lightening compositions according to the disclosure provide very high or even ultra-high levels of lift while minimizing the damaging effects of bleaching and/or of oxidizing agents in the compositions.

Hair lightening compositions according to the disclosure can be prepared by mixing, at or near the time of use, a base lightening composition according to the disclosure with a solvent to form a base lightening mixture, and combining the base lightening mixture with an oxidizing composition or oxidizing agent to form a hair lightening composition.

### Base Lightening Compositions

Base lightening compositions according to the disclosure include (i) at least one persulfate compound, for example at least two persulfate compounds, (ii) one or more silicate compounds, (iii) one or more alkanolamines and/or derivatives, (iv) one or more carboxylic acids and/or salts, and may optionally further one or more compounds chosen from alkalizing agents other than persulfates, oxidizing agents, thermal control agents, amino sulfonic acids, oils, anionic surfactants, fillers, thickeners, or combinations of two or more thereof.

The base lightening compositions may be in various forms, including but not limited to powder, cream, lotion, or gel. In at least some embodiments, the base lightening compositions are anhydrous or substantially anhydrous, for example comprise less than 3%, such as less than 2%, less than 1.5%, less than 1%, less than 0.5%, less than 0.1%, or less than 0.01% water by weight, relative to the total weight of the base lightening composition. In some embodiments, the base lightening compositions are substantially or entirely pulverulent.

### Persulfate Compounds

Base lightening compositions according to the disclosure comprise at least one persulfate compound, for example at least two persulfate compounds. In a particular embodiment, the base lightening compositions comprise (a) ammonium persulfate, and (b) at least one additional persulfate compound, for example potassium persulfate and/or sodium persulfate.

The total amount of persulfate compounds in base lightening compositions according to the disclosure is less than found in typical base lightening compositions, but surprisingly hair lightening compositions according to the disclosure are just as effective, and in some cases more effective, at lifting or lightening the color of the hair, even when very high or ultra-high levels of lift are desired. In various embodiments, the base lightening compositions will comprise less than about 50% persulfate compounds, such as no more than 50%, no more than 49%, no more than 48%, no more than 47%, no more than 46%, or no more than 45% persulfate compounds by weight, relative to the total weight of the base lightening composition. For example, base lightening compositions according to the disclosure typically comprise a total amount of persulfate compounds ranging from at least about 10%, such as at least about 15%, at least about 20%, at least about 25%, at least about 30%, at least about 35%, or at least about 40%, but less than about 50%, or less than about 45% by weight, relative to the total weight of the base lightening composition, including all ranges and subranges thereof. By way of non-limiting example, the total amount of persulfate compounds in the base lightening compositions may range from about 10% to about 50%, about 10% to about 45%, about 10% to about 40%, about 10% to about 35%, about 10% to about 30%, about 10% to about 25%, about 15% to about 50%, about 15% to about 45%, about 15% to about 40%, about 15% to about 35%, about 15% to about 30%, about 15% to about 25%, about 20% to about 50%, about 20% to about 45%, about 20% to about 40%, about 20% to about 35%, about 20% to about 30%, about 20% to about 25%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, or about 35% to about 40% by weight, relative to the total weight of the base lightening composition. As further examples, the total amount of persulfates may range from about 30% to about 40%, such as from about 31% to about 40%, from about 32% to about 40%, from about 33% to about 40%, about 30% to about 39%, from about 31% to about 39%, from about 32% to about 39%, from about 33% to about 38%, about 30% to about 38%, from about 31% to about 38%, from about 32% to about 38%, from about 33% to about 38%, about 30% to about 37%, from about 31% to about 37%, from about 32% to about 37%, or from about 33% to about 37% by weight, relative to the total weight of the base lightening composition. In some embodiments, the total amount of persulfates may be about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% by weight, relative to the total weight of the base lightening composition, including all ranges and subranges using any of the foregoing as upper and lower limits.

In embodiments where the base lightening composition comprises (a) ammonium persulfate and (b) at least one additional persulfate compound, the total amount of (a) ammonium persulfate in the base lightening composition can range from at least about 5%, such as at least about 6%, at least about 7%, at least about 8%, at least about 9%, at least about 10%, but less than about 20%, less than about 19%, less than about 18%, less than about 17%, less than about 16%, or less than about 15% by weight, relative to the total weight of the base lightening composition, including all ranges and subranges thereof. In some embodiments, the total amount of (a) ammonium persulfate may be about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the base lightening composition, including all ranges and subranges using any of the foregoing as upper and lower limits.

In embodiments where the base lightening composition comprises (a) ammonium persulfate and (b) at least one additional persulfate compound, the total amount of (b) additional persulfate compounds in the base lightening composition can range from at least about 15%, such as at least about 16%, at least about 17%, at least about 18%, at least about 19%, at least about 20%, at least about 22%, at least about 23%, at least about 24%, or at least about 25%, but less than about 40%, less than about 35%, or less than about 30% by weight, relative to the total weight of the base lightening composition, including all ranges and subranges thereof. In some embodiments, the total amount of (b) additional persulfate compounds may be about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight, relative to the total weight of the base lightening composition, including all ranges and subranges using any of the foregoing as upper and lower limits.

In various embodiments, it may be particularly advantageous to choose an amount of (a) ammonium persulfate that is equal to or less than the total amount of (b) additional persulfate compounds, for example less than the total amount of potassium persulfate and/or sodium persulfate. As non-limiting examples, the base lightening compositions may comprise ammonium persulfate and at least one additional persulfate compound chosen from potassium persulfate and/or sodium persulfate, where the weight ratio of (a) the amount of ammonium persulfate to (b) the total amount of additional persulfate compounds, for example potassium sulfate, or the weight ratio of the amount of ammonium persulfate to the total amount of additional persulfate compounds, for example potassium persulfate and sodium persulfate, is equal to or less than about 1. By way of example, the weight ratio may range from about 0.1 to about 0.9, such as from about 0.2 to about 0.8, from about 0.3 to about 0.7, or from about 0.4 to about 0.6. In some embodiments, the weight ratio of (a) the amount of ammonium persulfate to (b) the total amount of additional persulfate compounds, for example potassium persulfate and/or sodium persulfate, is about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, or about 0.9, or may be any range using any of the foregoing as upper and lower limits.

### Silicate Compounds

Base lightening compositions according to the disclosure comprise at least one silicate compound. Non-limiting examples of silicates include lithium, sodium, and potassium silicates, metasilicates, and disilicates, and combinations of two or more thereof, including mixed lithium, sodium, and potassium salts thereof. Specific non-limiting examples include aluminum silicate, magnesium silicate, aluminum magnesium silicate, calcium silicate, barium silicate, strontium silicate, potassium silicate, potassium metasilicate, sodium silicate, sodium metasilicate, or any combination of two or more thereof. In at least some embodiments, the base lightening compositions comprise at least one metasilicate compound, for example sodium metasilicate. In some embodiments the base lightening compositions comprise at least two silicate compounds, wherein at least one is a metasilicate.

The total amount of silicate compounds in base lightening compositions according to the disclosure may, in at least some embodiments, be greater than found in typical base lightening compositions. In particular, it may be advantageous in some embodiments to increase the amount of silicate compounds in base lightening compositions according to the disclosure to achieve and/or maintain a desired pH of the hair lightening compositions.

For example, the total amount of silicate compounds may range from about 5% to about 50%, such as from about 15% to about 45%, or from about 30% to about 40% by weight, based on the total weight of the base lightening composition. In various embodiments, the total amount of silicate compounds may range from about 5% to about 45%, about 5% to about 40%, about 5% to about 35%, about 5% to about 20%, about 10% to about 50%, about 10% to about 45%, about 10% to about 40%, about 10% to about 35%, about 10% to about 30%, about 15% to about 50%, about 15% to about 45%, about 15% to about 40%, about 15% to about 35%, about 15% to about 30%, about 20% to about 50%, about 20% to about 45%, about 20% to about 40%, about 20% to about 35%, about 20% to about 30%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, or about 35% to about 40% by weight, based on the total weight of the base lightening composition.

### Benefit Agents

Base lightening compositions according to the disclosure comprise at least one benefit agent for strengthening or protecting the integrity of the hair fiber during lightening treatments. Benefit agents according to the disclosure are chosen from amine compounds. Salts and derivatives of amine compounds can be used, and are expressly included unless otherwise stated. Useful salts include but are not limited to chloride salts. Amine compounds that can be used as benefit agents are chosen from compounds of formula (I):

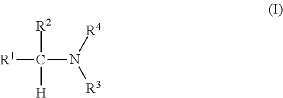

wherein:
R¹ is chosen from —C(O)OH, —CH₂C(O)OH, —(CH₂)₃N(H)C(NH₂)₂, a C1-C4 alkyl radical, or a C1-C4 hydroxyalkyl radical;
R² is chosen from hydrogen, a C1-C4 hydroxyalkyl radical, or R⁵CH₂OH;
R³ and R⁴ are independently chosen from hydrogen or a C1-C4 hydroxyalkyl radical; and
R⁵ is a C1-C4 alkyl radical.

In various embodiments, R³ and R⁴ are identical, for example hydrogen or (CH₂)₂OH. In various embodiments, R¹ is chosen from —C(O)OH or a C1-C4 hydroxyalkyl radical, preferably a C1-C2 hydroxyalkyl radical. In embodiments where R³ and R⁴ are both (CH₂)₂₀H, R¹ can, for example, be chosen from —C(O)OH or a C1-C4 hydroxyalkyl radical, e.g. CH₂OH or (CH₂)₂₀H. In embodiments where R³ and R⁴ are both hydrogen, R¹ is preferably —C(O)OH. Useful benefit agents include amino acids of formula (I) and their derivatives.

As non-limiting examples of benefit agents, alkanolamines such as monoethanolamine, diethanolamine, or triethanolamine can be used. Amino acids of formula (I) such as aspartic acid, serine, threonine, arginine, alanine, or glycine can also be chosen. Derivatives of those amino acids, for example N,N-bis(2-hydroxyethyl)glycine (bicine), can also be used.

The total amount of benefit agents in the base lightening composition can range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, or about 7% by weight, relative to the total weight of the base lightening composition, including all ranges and subranges using any of the foregoing as upper and lower limits.

### Carboxylic Acids

Base lightening compositions according to the disclosure comprise at least one carboxylic acid and/or carboxylic acid salt. Salts of carboxylic acids are expressly included unless otherwise stated. Useful salts include but are not limited to sodium and potassium salts. In preferred embodiments, base lightening compositions comprise one or more salts of carboxylic acids and are free or substantially free of carboxylic acids.

The carboxylic acids can be chosen from non-polymeric mono-, di-, and/or tri-carboxylic acids. Non-limiting examples of useful carboxylic acids include citric acid, succinic acid, aspartic acid, glutamic acid, lactic acid, malic acid, tartaric acid, glutaric acid, acetic acid, glycolic acid, oxalic acid, salts thereof, or combinations thereof. In some embodiments, the carboxylic acid chosen comprises, consists essentially of, or consists of citric acid and salts thereof, such as sodium citrate. In preferred embodiments, sodium citrate is used.

The total amount of carboxylic acids and salts thereof may range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, or about 7% by weight, relative to the total weight of the base lightening composition, including all ranges and subranges using any of the foregoing as upper and lower limits.

In at least some embodiments, the base lightening compositions comprise one or more carboxylic acid salts, but are free or essentially free of carboxylic acids. For example, the base lightening compositions may comprise less than about 1%, such as less than about 0.9%, less than about 0.8%, less than about 0.7%, less than about 0.6%, less than about 0.5%, less than about 0.4%, less than about 0.3%, less than about 0.2%, less than about 0.1%, less than about 0.075%, less than about 0.05%, less than about 0.025%, less than about 0.01%, less than about 0.001%, or less than about 0.0001% of carboxylic acids, without regard to salts of carboxylic acids. Unless otherwise specified, “substantially free of carboxylic acids” means that the base lightening composition contains less than about 0.01% of carboxylic acids, without regard to salts of carboxylic acids.

It has been surprisingly discovered that by choosing amounts of benefit agents and carboxylic acids and/or salts relative to each other, hair lightening compositions according to the disclosure can provide very high or even ultra-high levels of lift and minimize damage to the hair that would otherwise be expected from compositions and methods for achieving such high levels of lift. Therefore, in various embodiments, the amounts of benefit agents and carboxylic acids and/or salts are chosen such that the weight ratio of benefit agents to carboxylic acids and/or salts in the base lightening composition and/or the hair lightening composition ranges from about 0.2 to about 2, such as from about 0.3 to about 1.8, from about 0.4 to about 1.6, from about 0.5 to about 1.5, from about 0.6 to about 1.4, from about 0.7 to about 1.3, from about 0.8 to about 1.2, from about 0.9 to about 1.1, or is about 1.

In particularly preferred embodiments, base lightening compositions according to the disclosure comprise at least one benefit agent and at least one carboxylic acid salt, for example at least one salt of citric acid such as sodium citrate, wherein the weight ratio of benefit agents to carboxylic acid salts ranges from about 0.2 to about 2, such as from about 0.3 to about 1.8, from about 0.4 to about 1.6, from about 0.5 to about 1.5, from about 0.6 to about 1.4, from about 0.7 to about 1.3, from about 0.8 to about 1.2, from about 0.9 to about 1.1, or is about 1. In such embodiments, the benefit agents may, for example, be chosen from alkanolamines and/or amino acids and derivatives thereof of formula (I), as well as salts thereof.

### Alkalizing Agents

The base lightening compositions according to the disclosure may optionally further comprise at least one alkalizing agent other than silicates. The alkalizing agents may be chosen from organic and/or mineral alkalizing agents, for example, organic amines and their organic or mineral salts, ammonium salts, and mineral alkalizing agents such as inorganic bases and hydroxide base compounds.

In various embodiments, it may be advantageous to include amounts of alkalizing agents in the base lightening compositions to achieve and/or maintain a desired pH of the hair lightening compositions.

### Organic Alkalizing Agents

As non-limiting examples, suitable organic alkalizing agents may be chosen from compounds of formula (II):

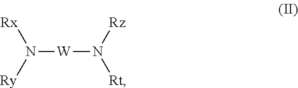

wherein:
W is chosen from C1-C6 alkylene residues optionally substituted with a hydroxyl group or a C1-C6 alkyl radical; and
Rx, Ry, Rz, and Rt, which may be identical or different, are chosen from a hydrogen atom, C1-C6 alkyl radicals, C1-C6 hydroxyalkyl radicals, and C1-C6 aminoalkyl radicals.

By way of example, compounds of formula (II) may be chosen from 1,3-diaminopropane, 1,3-diamino-2-propanol, spermine, and spermidine.

Useful organic salts include, by way of example only, citrates, lactates, glycolates, gluconates, acetates, propionates, fumarates, oxalates, and tartrates. Useful mineral salts include, by way of example only, hydrohalides (for example hydrochlorides), carbonates, hydrogen carbonates, sulfates, hydrogen phosphates, and phosphates.

Non-limiting examples of ammonium salts that may be used include carbonate and bicarbonate.

If present, the total amount of organic alkalizing agents may independently range from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4% by weight, based on the total weight of the base lightening composition.

### Mineral Alkalizing Agents

Useful and non-limiting mineral alkalizing agents include metal hydroxides and carbonates. Suitable metal hydroxides and carbonates include alkali metal and alkaline earth metal hydroxides or carbonates. Examples of such metal hydroxides include sodium, potassium, lithium, calcium, magnesium. In some embodiments, the alkalizing agent comprises ammonia, ammonium carbonates, sodium carbonates, potassium carbonates, ammonium bicarbonates, sodium bicarbonates, potassium bicarbonates, ammonium hydroxides, sodium hydroxides, potassium hydroxides, salts thereof, hydrates thereof, or mixtures thereof. In some embodiments, potassium carbonate may be used.

If present, the total amount of mineral alkalizing agents may range from about 0.1% to about 10% by weight, relative to the total weight of the base lightening composition, including all subranges therebetween. For example, the base lightening composition may contain from about 0.5% to about 10%, from about 0.75% to about 9%, from about 1% to about 8.5%, from about 1.5% to about 8%, from about 1.75% to about 7.5%, from about 2% to about 7%, from about 2.25% to about 6.5%, from about 2.5% to about 6%, from about 2.75% to about 5.5%, from about 3% to about 5.25%, or from about 3% to about 5% by weight, based on the total weight of the base lightening composition, including all ranges using any of the foregoing as upper and lower limits.

### Thermal Control Agents

It is known that when a base lightening composition is mixed with an aqueous developer to form a hair lightening composition, a chemical reaction occurs that raises the temperature of the mixture, for example to temperatures greater than about 70° C. (i.e. greater than about 160° F.), which can result in conditions that are uncomfortable for the user and also potentially unsafe. Therefore, in various embodiments the base lightening composition may optionally include one or more thermal control agents, for example sodium stearate, urea, mineral oil, magnesium carbonate, and/or magnesium carbonate hydroxide.

If present, the total amount of thermal control agents may vary, but typically ranges from about 0.1% to about 20%, such as from about 3% to about 18%, from about 5% to about 15%, or from about 8% to about 12%, or may be about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the base lightening composition, including all ranges and subranges using any of the foregoing as upper and lower limits.

For example, the total amount of urea may range from about 0.1% to about 15%, such as from about 0.5% to about 10%, from about 0.5% to about 8%, from about 0.5% to about 6%, from about 0.5% to about 5%, from about 0.5% to about 4%, from about 0.5% to about 3%, from about 0.5% to about 2%, from about 1% to about 10%, from about 1% to about 8%, from about 1% to about 6%, from about 1% to about 5%, from about 1% to about 4%, from about 1% to about 3%, from about 1% to about 2%, from about 1.5% to about 15%, from about 1.5% to about 10%, from about 1.5% to about 8%, from about 1.5% to about 6%, from about 1.5% to about 5%, from about 1.5% to about 4%, from about 1.5% to about 3%, from about 2% to about 10%, from about 2% to about 8%, from about 2% to about 6%, from about 2% to about 5%, from about 2% to about 4%, from about 2% to about 3%, from about 2.5% to about 10%, from about 2.5% to about 8%, from about 2.5% to about 6%, from about 2.5% to about 5%, from about 2.5% to about 4%, from about 2.5% to about 3%, from about 3% to about 10%, from about 3% to about 8%, from about 3% to about 6%, from about 3.5% to about 5%, from about 3.5% to about 4%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the base lightening composition, including all ranges and subranges using any of the foregoing as upper and lower limits.

As a further example, the base lightening compositions may comprise from about 0.1% to about 10%, such as from about 0.5% to about 7.5%, from about 1% to about 5%, or from about 1.5% to about 3.5% sodium stearate by weight, relative to the total weight of the base lightening composition. In some embodiments sodium stearate and mineral oil can be used together to work synergistically to control the temperature of the lightening composition without negatively impacting the level of lightening achieved. This surprising effect is most prominently seen when the weight ratio of sodium stearate to mineral oil ranges from about 1:3 to about 3:1, such as from about 1:2.5 to about 2.5:1, from about 1:2 to about 2:1, from about 1:1.5 to about 1.5:1, from about 1:1.25 to about 1.25:1, or from about 1:1.1 to about 1.1:1, or is about 1:1. If present, the amounts of sodium stearate and mineral oil may vary, but may, for example, independently range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, or about 5% by weight, relative to the total weight of the base lightening composition, including all ranges and subranges using any of the foregoing as upper and lower limits.

### Oxidizing Agents

Base lightening compositions according to the disclosure optionally comprise at least one oxidizing agent other than persulfates. Useful and non-limiting examples of oxidizing agents include perborates, percarbonates, peroxides, organic peracids, salts thereof, or mixtures thereof. In some embodiments, the base lightening compositions can include, as oxidizing agents, one or more of alkali metal bromates, ferricyanides, redox enzymes such as laccases, peroxidases, solid hydrogen peroxides such as urea hydrogen peroxide, sodium peroxide, and strontium peroxide, and 2-electron oxidoreductases, such as uricase.

If present, the total amount of oxidizing agents other than persulfates in the base lightening compositions may, in various embodiments, range from about 0.5% to about 45%, such as from about 1% to about 40%, from about 1.5% to about 35%, from about 2% to about 30%, from about 2.5% to about 25%, from about 3% to about 20%, from about 3.5% to about 15%, from about 4% to about 12%, from about 4.5% to about 10%, or from about 5% to about 8% by weight, based on the total weight of the hair base lightening composition, including all ranges using any of the foregoing as upper and lower limits. In at least some embodiments, the base lightening composition is free or substantially free of oxidizing agents other than persulfates.

### Oils

In some cases, the base lightening compositions include one or more oils. Non-limiting examples of oils include ester oils, silicone oils, fluoro oils, vegetable oils, animal oils, hydrocarbon-based oils, or mixtures thereof. For example, lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, shea butter, or combinations of two or more thereof can be used. Mention is made, as an example of linear or branched hydrocarbons, of oils of mineral or synthetic origin, such as liquid paraffins and their derivatives, petrolatum, mineral oils, (mixtures of oil-derived hydrocarbon oils), polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane, and their mixtures.

The total amount of oils can vary, but typically ranges from about 0.01% to about 10%, such as from about 0.1% to about 8%, or from about 0.5% to about 6% by weight, relative to the total weight of the base lightening composition. For example, in some embodiments, the total amount of oils may range from about 0.5% to about 5%, from about 0.5% to about 4%, from about 0.5% to about 3%, from about 0.5% to about 2.5%, from about 0.5% to about 2%, from about 0.5% to about 1.5%, from about 0.5% to about 1%, from about 0.75% to about 5%, from about 0.75% to about 4%, from about 0.75% to about 3%, from about 0.75% to about 2.5%, from about 0.75% to about 2%, from about 0.75% to about 1.5%, from about 0.75% to about 1%, from about 1% to about 5%, from about 1% to about 4%, from about 1% to about 3%, from about 1% to about 2.5%, from about 1% to about 2%, or from about 1% to about 1.5% by weight, relative to the total weight of the base lightening composition.

### Fillers

Optionally, base lightening compositions according to the disclosure comprise at least one filler (other than silicates). Non-limiting examples of fillers include starches, maltodextrins, perlites, zeolites, polylactic acids, silicas, polyamide powders, polyvinylpyrrolidones, dextrose, oligosaccharides, celluloses, diatomite, diatomaceous earth, talc, clays, silicon dioxide, clays, or mixtures thereof. In some instances, the one or more fillers comprises one or more alkali metal salts of fatty acids and/or organic base salts of fatty acids, for example, sodium stearate, zinc laurate, magnesium stearate, magnesium myristate, zinc stearate, ammonium stearate, ammonium oleate, ammonium nonanoate, sodium palmitate, potassium stearate, potassium palmitate, sodium myristate, aluminum monostearate, or combinations of two or more thereof.

If present, the total amount of fillers may vary, but typically ranges from about 1% to about 20%, including all subranges therebetween, such as from about 1% to about 18%, from about 2% to about 16%, from about 2.5% to about 15%, or from about 3% to about 14%, or from about 4% to about 13% by weight, relative to the total weight of the base lightening composition, for example from about 1% to about 18%, from about 1% to about 16%, from about 1% to about 15%, from about 1% to about 14%, from about 1% to about 13%, from about 1% to about 12%, from about 1.5% to about 20%, from about 1.5% to about 16%, from about 1.5% to about 15%, from about 1.5% to about 14%, from about 1.5% to about 13%, from about 1.5% to about 12%, from about 2% to about 20%, from about 2% to about 18%, from about 2% to about 15%, from about 2% to about 14%, from about 2% to about 13%, from about 2% to about 12%, from about 2.5% to about 20%, from about 2.5% to about 18%, from about 2.5% to about 16%, from about 2.5% to about 14%, from about 2.5% to about 13%, from about 2.5% to about 12%, from about 3% to about 20%, from about 3% to about 18%, from about 3% to about 16%, from about 3% to about 15%, from about 3% to about 13%, from about 3% to about 12%, from about 4% to about 20%, from about 4% to about 18%, from about 4% to about 16%, from about 4% to about 15%, from about 4% to about 14%, or from about 4% to about 12% by weight, relative to the total weight of the base lightening composition.

### Thickening Agents

The base lightening compositions can also optionally include one or more thickening agents. Non-limiting examples of thickening agents include carboxylic acid polymers, crosslinked polyacrylate polymers, polyacrylamide polymers, polysaccharides, and gums. More specific, non-limiting examples of thickening agents include acacia, agar, algin, alginic acid, ammonium alginate, amylopectin, calcium alginate, calcium carrageenan, carnitine, carrageenan, celluloses and modified celluloses such as hydroxyethylcellulose, dextrin, gelatin, gellan gum, guar gum, guar hydroxypropyltrimonium chloride, hydroxylpropyl guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, or mixtures thereof. In some cases, the one or more thickening agents include a crosslinked polyacrylate polymer, for example, acrylates/C10-30 alkylacrylate crosspolymer. Furthermore, in some cases the one or more thickening agents include guar gum.

If present, the total amount of thickening agents may range from about 0.01% to about 10%, such as from about 0.01% to about 5%, from about 0.1% to about 5%, from about 0.5% to about 5%, from about 0.5% to about 3%, from about 1% to about 5%, from about 1% to about 3%, from about 1.25% to about 2.75%, or from about 1.5% to about 2.5% by weight, relative to the total weight of the base lightening composition.

### Anionic Surfactants

Optionally, base lightening compositions comprise one or more anionic surfactants. For instance, the anionic surfactant(s) that may be useful include alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

The salts of C6-C24 alkyl monoesters of polyglycoside-polycarboxylic acids may be chosen from C6-C24 alkyl polyglycoside-citrates, C6-C24 alkyl polyglycoside-tartrates and C6-C24 alkyl polyglycoside-sulfo succinates.

Use is also made of (C6-C24)alkyl sulfates, (C6-C24)alkyl ether sulfates, which are optionally ethoxylated, comprising from 2 to 50 ethylene oxide units, or mixtures thereof, in particular in the form of alkali metal salts or alkaline-earth metal salts, ammonium salts or amino alcohol salts.

In some cases, the one or more anionic surfactant is selected from the group consisting of sodium oleyl succinate, ammonium lauryl sulphosuccinate, sodium lauryl sulfate, sodium lauryl ether sulfate (also known as sodium laureth sulfate, SLES), sodium lauryl ether sulphosuccinate, ammonium lauryl sulfate (ALS), ammonium lauryl ether sulfate (ammonium laureth sulfate), sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate, or mixtures thereof. In some instances, preferred anionic surfactants include sodium lauryl sulfate, sodium lauryl ether sulfate, sodium lauryl ether sulphosuccinate, ammonium lauryl sulfate, ammonium lauryl ether sulfate, or mixtures thereof.

If present, the total amount of anionic surfactants may range from about 0.1% up to about 15%, such as up to about 12%, up to about 10%, up to about 9%, up to about 8%, up to about 7%, or up to about 6% by weight, relative to the total weight of the base lightening composition. For example, the total amount of anionic surfactants may range from about 0.1% to about 10%, from about 0.5% to about 9%, from about 1% to about 8%, or from about 2% to about 7% by weight, relative to the total weight of the base lightening composition. In at least some embodiments, the base lightening compositions comprise at least one anionic surfactant, and have a total amount of anionic surfactants ranging from about 0.5% to about 12%, such as from about 1% to about 10%, from about 1.5% to about 9%, or from about 2% to about 8%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the base lightening composition, including any range using any of the foregoing as upper and lower limits.

The base lightening composition may comprise additional auxiliary components, for example preservatives, hair conditioning agents, vitamins, chelating agents, pigments or colorants to color the composition, or any other component typically used in hair color lightening base compositions.

In some embodiments the base lightening composition will be anhydrous or substantially anhydrous, for example with less than about 1% water, such as less than about 0.5% water, or less than about 0.1% water. The form of the base lightening composition may be a powder, cream, gel, or the like.

### Base Lightening Mixtures

As discussed, the base lightening compositions use less persulfates than traditional base lightening compositions, which typically comprise at least 50% or more persulfates. However, commercially-available developer compositions are formulated to be mixed in predetermined mix ratios with traditional base lightening compositions. Therefore, in some embodiments the base lightening compositions according to the disclosure can be mixed with an appropriate amount of a solvent, such as water, to form a base lightening mixture. The base lightening mixture can then be mixed with an oxidizing composition or oxidizing agent in a desired mix ratio.

The choice of solvent is not limited other than that it will generally not significantly reduce the lightening effects, slow the lightening process, or otherwise interfere with the intended result of the process. Unexpectedly, it has been discovered that by using a solvent that comprises, consists essentially of, or consists of water a greater increase in lift can be achieved. This finding was particularly surprising as adding water would have been expected to dilute or diminish the bleaching power. As such, it may be particularly advantageous to use water as the solvent. Therefore, in various embodiments, the solvent comprises, consists essentially of, or consists of water.

As another example, the solvent can be an aqueous oxidizing (developer) composition. The choice of aqueous oxidizing composition as a solvent to prepare a base lightening mixture is not limited, and can, for example, include any of the aqueous oxidizing compositions described below. In addition, in various embodiments it may be advantageous to choose a type and/or strength of developer composition as solvent which is the same or different than that used to prepare the hair lightening composition. For example, the developer chosen as a solvent for preparing the base lightening mixture may be a 10V, 20V, 30V, or 40V aqueous hydrogen peroxide composition, which may or may not be the same as the oxidizing composition used to prepare the hair lightening composition.

The amount of solvent to be mixed with or added to the base lightening composition to prepare the base lightening mixture can be determined by the amount of persulfates and alkalizers present. Typically, traditional base lightening compositions will comprise in the range of 50-60% persulfates. Thus, for example, a base lightening composition comprising 35% persulfates and an additional 5% of alkalizers could be mixed with an amount of solvent in the range of 10-20% by weight, relative to the total weight of the base lightening composition, as a superaddition.

In various embodiments, the amount of solvent that may be mixed with or added to the base lightening composition can range from about 1% to about 25%, such as, for example, from about 1% to about 20%, from about 1.5% to about 18%, from about 2% to about 15%, from about 2.5% to about 12%, from about 3% to about 10%, from about 3.5% to about 8%, from about 4% to about 6%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 20.5%, about 21%, about 21.5%, about 22%, about 22.5%, about 23%, about 23.5%, about 24%, about 24.5%, or about 25% by weight, relative to the total weight of the base lightening composition, including all ranges and subranges using any of the foregoing as upper and lower limits.

In other embodiments, the amount of solvent added to or mixed with the base lightening composition may be chosen relative to the total amount of persulfates in the base lightening composition. For example, the weight ratio of total persulfates in the base lightening composition to the amount of solvent may range from about 1:1 to about 10:1, such as from about 1:1 to about 5:1, or from about 1.5:1 to about 3.5:1. In various embodiments, the weight ratio of the total amount of persulfates in the base lightening composition to the amount of solvent added to or mixed with the base lightening composition may be about 1:1, about 1.25:1, about 1.5:1, about 1.75:1, about 2:1, about 2.25:1, about 2.5:1, about 2.75:1, about 3:1, about 3.25:1, about 3.5:1, about 3.75:1, about 4:1, about 4.25:1, about 4.5:1, about 4.75:1, or about 5:1, or may be any range using any of the foregoing as upper and lower limits.

In other embodiments still, the amount of solvent added to or mixed with the base lightening composition may be chosen relative to the total weight of the base lightening composition. For example, the weight ratio of total weight of the base lightening composition to solvent may range from about 5:1 to about 15:1, such as from about 6:1 to about 14:1, or from about 7:1 to about 13.5:1. In various embodiments, the weight ratio of the total weight of the base lightening composition to the amount of solvent added to or mixed with the base lightening composition may be about 5:1, about 5.25:1, about 5.5:1, about 5.75:1, about 6:1, about 6.25:1, about 6.5:1, about 6.75:1, about 7:1, about 7.25:1, about 7.5:1, about 7.75:1, about 8:1, about 8.25:1, about 8.5:1, about 8.75:1, about 9:1, about 9.25:1, about 9.5:1, about 9.75:1, about 10:1, about 10.25:1, about 10.5:1, about 10.75:1, about 11:1, about 11.25:1, about 11.5:1, about 11.75:1, about 12:1, about 12.25:1, about 12.5:1, about 12.75:1, about 13:1, about 13.25:1, about 13.5:1, about 13.75:1, about 14:1, about 14.25:1, about 14.5:1, about 14.75:1, or about 15:1, or may be any range using any of the foregoing as upper and lower limits

Although not necessary, it may be advantageous for the user to combine the base lightening composition and solvent to prepare the base lightening mixture at or near the time of use, for example just before or simultaneously with mixing the base lightening composition and oxidizing composition/oxidizing agent. It should be understood that the order of mixing is not critical. Alternatively, the solvent may be added to the base lightening composition during manufacture.

### Oxidizing Compositions and Oxidizing Agents

The oxidizing composition (developer) or oxidizing agent that can be mixed with the base lightening composition or mixture to prepare the hair lightening composition at or near the time of use is not limited, and any conventional oxidizing composition or oxidizing agent for lightening hair can be used. Typically an oxidizing composition comprises an oxidizing agent such as peroxide, inorganic perhydrate salts, or a source of peroxymonocarbonate ions, in a cosmetically acceptable carrier. Non-limiting examples of peroxides include hydrogen peroxide, inorganic alkali metal peroxides such as sodium peroxide, and organic peroxides such as urea peroxide and melamine peroxide. Non-limiting examples of inorganic perhydrate salts include the alkali metal salts of perborates, percarbonates, perphosphates, persilicates, persulphates, and the like. These inorganic perhydrate salts may optionally be used as monohydrates, tetrahydrates, etc.

As non-limiting examples of sources of peroxymonocarbonate ions, in situ formation from a source of hydrogen peroxide and a source of hydrogen carbonate ions can be used. This system is particularly effective in combination with a source of ammonia or ammonium ions. Accordingly, any source of peroxymonocarbonate ions may be utilized. Suitable sources include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, barium carbonate, ammonium carbonate, ammonium hydrogen carbonate, and mixtures thereof. Percarbonate salts may also be utilized to provide both the source of carbonate ions and as an oxidizing agent. Non-limiting examples of carbonate ions, carbamate, and hydrocarbonate ions are sodium hydrogen carbonate, potassium hydrogen carbonate, ammonium carbamate, and mixtures of two or more thereof.

The amount of oxidizing agent will vary, but typically ranges from about 1% to about 40%, such as from about 2% to about 30%, from about 2% to about 25%, from about 2% to about 20%, from about 2% to about 15%, from about 2% to about 10%, from about 2% to about 5%, from about 5% to about 30%, from about 5% to about 25%, from about 5% to about 20%, from about 5% to about 15%, from about 5% to about 10%, from about 10% to about 30%, from about 10% to about 25%, from about 10% to about 20%, or from about 10% to about 15% by weight, relative to the total weight of the oxidizing composition, including any range using any of the foregoing as upper and lower limits. By way of example only, the oxidizing agent may comprise, consist essentially of, or consist of hydrogen peroxide, present in any of the foregoing ranges, for example about 3%, about 6%, about 12%, or about 18% by weight, relative to the total weight of the oxidizing composition. However, because oxidizing agents also damage the hair, it would be advantageous to reduce the amount needed to achieve the same or even greater levels of lightening.

The oxidizing composition may further comprise additional components such as thermal control agents as described above, oils as described above, anionic surfactants as described above, thickening agents as described above, amino sulfonic acids and their salts, nonionic surfactants, cationic surfactants, amphoteric surfactants, hair conditioning agents, preservatives, vitamins, chelating agents, pigments or colorants to color the composition, pH adjusters, fragrances, or any other component typically used in developer compositions for hair lightening.

The oxidizing composition may be in the form of a powder, gel, liquid, foam, lotion, cream, mousse, or emulsion. In some embodiments, the oxidizing composition is anhydrous, and is mixed with water prior to or simultaneously with being mixed with a base lightening composition or mixture. The oxidizing composition may optionally be aqueous, and have a pH less than 7, such as ranging from about 1 to about 6 or about 2 about 5. For example, the oxidizing composition may be a 10V, 20V, 30V, or 40V aqueous hydrogen peroxide composition, and have a pH ranging from about 2.5 to about 4.5.

### Hair Lightening Compositions

Hair lightening compositions comprise the components of base lightening compositions according to the disclosure, optionally one or more solvents that is (are) mixed with the base lightening composition, and an oxidizing agent. Typically, a base lightening composition and an oxidizing composition (developer) are mixed at or near the time of use in a predetermined mix ratio, depending on strength and level of lift to be achieved. Such predetermined ratios typically range from about 1:3 to about 3:1, such as from about 1:2.5 to about 2.5:1, from about 1:2 to about 2:1, or from about 1:1.5 to about 1.5:1, for example about 1:1.

In embodiments where one or more solvents is mixed with the base lightening composition to form a base lightening mixture, the base lightening mixture and oxidizing composition comprising the oxidizing agent may be mixed at or near the time of use, for example in a mix ratio of [base lightening mixture]:[oxidizing composition] ranging from about 1:3 to about 3:1, such as from about 1:2.5 to about 2.5:1, from about 1:2 to about 2:1, or from about 1:1.5 to about 1.5:1, such as about 1:1, to form the hair lightening composition. For example, the mix ratio of [base lightening mixture]:[oxidizing composition] may be about 1:1, about 1:1.5, or about 1:2.

In embodiments where an additional amount of oxidizing composition is used as a solvent, a base lightening mixture may, in some cases, not be prepared prior to forming the hair lightening composition; rather, the base lightening composition may be mixed directly with the oxidizing composition. In such embodiments, a base lightening composition is mixed with the oxidizing composition in an “effective mix ratio” that can be determined by adding the amount of base lightening composition and the amount of additional oxidizing composition used, and subtracting the amount of additional oxidizing composition from the total amount of oxidizing composition. Thus, an “effective mix ratio” can be calculated as [base lightening composition+additional oxidizing composition]:[total oxidizing composition−additional oxidizing composition]. Exemplary effective mix ratios may range from about 1:3 to about 3:1, such as from about 1:2.5 to about 2.5:1, from about 1:2 to about 2:1, or from about 1:1.5 to about 1.5:1, such as about 1:1, to form the hair lightening composition. For example, the effective mix ratio of [base lightening composition+additional oxidizing composition]:[total oxidizing composition−additional oxidizing composition] may be about 1:1, about 1:1.5, or about 1:2.

As such, the amounts of components described herein for the base lightening composition and oxidizing composition will be present in the hair lightening composition in amounts consistent with the chosen mix ratio. By way of illustration only, if a 1:1 mix ratio of [base lightening mixture]:[oxidizing composition] is used, the amounts of components in the oxidizing composition will be present in the hair lightening composition at 50% of the amounts described herein. The amounts of components in the base lightening composition will likewise be present in the hair lightening composition at 50% of the amounts described herein for the base lightening composition, less the amount of solvent added.

As previously indicated, it may be particularly advantageous for hair lightening compositions according to the disclosure to comprise one or more carboxylic acid salts, but be free or essentially free of carboxylic acids. For example, the hair lightening compositions may comprise less than about 1%, such as less than about 0.9%, less than about 0.8%, less than about 0.7%, less than about 0.6%, less than about 0.5%, less than about 0.4%, less than about 0.3%, less than about 0.2%, less than about 0.1%, less than about 0.075%, less than about 0.05%, less than about 0.025%, less than about 0.01%, less than about 0.001%, or less than about 0.0001% of carboxylic acids, without regard to salts of carboxylic acids. Unless otherwise specified, “substantially free of carboxylic acids” means that the hair lightening composition contains less than about 0.01% of carboxylic acids, without regard to salts of carboxylic acids.

The hair lightening composition will typically have a pH ranging from about 8 to about 13, such as from about 9 to about 12, from about 9.5 to about 11, from about 10 to about 11, or from about 10 to about 10.5.

Hair lightening compositions according to the disclosure are surprisingly effective at providing very high or even ultra-high levels of lightening in a shorter period of time and/or using lower volumes of developer compared to traditional hair lightening compositions, despite the reduced amounts of persulfates. Because the exposure of the hair to the lightening composition is reduced and the compositions comprise one or more benefit agents, the treated hair also has less damage, is stronger, and appears and feels healthier compared to hair treated with traditional hair lightening compositions.

The hair lightening compositions are stable and have a smooth, light, whip cream-like texture upon mixing the base lightening composition or base lightening mixture and developer, which texture remains during the lightening process. This is in contrast to traditional hair lightening mixtures which become thick during application and solid and crumbly during the lightening process.

Hair lightening compositions according to the disclosure effectively control the peak temperature that is attained during the lightening process. In various embodiments, the peak temperature is less than 70° C., for example less than about 68° C., less than about 65° C., less than about 63° C., less than about 60° C., less than about 58° C., less than about 55° C., less than about 53° C., or less than about 50° C.

### II. Methods

The disclosure further relates to methods of lightening hair, as well as to methods of protecting hair from damage during a lightening process and/or methods of reducing damage to hair during a lightening process. The methods comprise mixing a base lightening composition according to the disclosure and an oxidizing composition at or near the time of use and applying the resulting mixture (hair lightening composition) to the hair. Surprisingly, compositions according to the disclosure have been found to not only effectively provide high, very high, or even ultra-high levels of lift, but at the same time minimize or prevent damage to the hair.

Methods of lightening the color of hair are known. Typically, after the base lightening composition and developer composition are mixed, the hair lightening composition is applied to the hair (e.g., by brush, foam applicator, etc.), optionally the treated hair is covered, e.g., with foil, the composition allowed to remain on the hair for a time sufficient to achieve the desired alteration in the color of the hair (processing time), and then the composition is removed from the hair, e.g., by wiping, rinsing, and/or shampooing the hair.

It may, in various embodiments, be advantageous to employ a single application of the hair lightening composition to the hair, for example when using medium or higher levels of oxidizing agents such as 30V or greater aqueous hydrogen peroxide developers (or equivalent), or to apply the hair lightening composition more than one time, for example when using medium or lower levels of oxidizing agents such as 30V or lower aqueous hydrogen peroxide developers (or equivalent). For example, if a single application is contemplated, the hair lightening composition may be applied to the hair, allowed to process for a desired processing time, and then the hair may be rinsed and/or shampooed. In embodiments where more than one application is contemplated, the hair lightening composition may be applied to the hair, allowed to process for a desired processing time, all or substantially all of the composition removed from the hair, a second application of the hair lightening composition applied to the hair and the hair allowed to process for a second desired processing time, and then the hair may be rinsed and/or shampooed. The first hair lightening composition can be removed from the hair by any method, for example wiping the hair, scraping the hair, rinsing the hair, etc.

In embodiments where more than one application of hair lightening composition is contemplated, the second application of hair lightening composition is understood to occur within the same service or treatment, e.g. within the same day (24 hours or less) as the first application, such as within less than 12 hours after the first application, within less than 6 hours after the first application, within less than 4 hours after the first application, within less than 3 hours after the first application, within less than 2 hours after the first application, or within less than 1 hour after the first application.

In embodiments where more than one application of hair lightening composition is contemplated, the hair lightening composition used in a second application may be the same or different as the first hair lightening composition, and/or the second processing time may be the same or different as the first processing time. It may, for example, be possible to use a second hair lightening composition according to the disclosure that is different than the first hair lightening composition according to the disclosure, or it may be possible to use a second hair lightening composition other than those described herein. It has been surprisingly discovered that hair lightening compositions according to the disclosure provide higher levels of lift when multiple applications of a hair lightening composition are employed compared to a single application, even when the overall processing times are the same. Therefore, a multiple application method can provide higher levels of lift than a single application method, and/or reduced processing times to achieve the same level of lift as a single application method.

It should also be understood that more than two applications of hair lightening composition can be employed in multiple application methods, following the same general protocol. For example, a third application of hair lightening composition, which may be the same as or different than the first and/or second hair lightening compositions may be applied to the hair, and the hair left to process for a processing time that is the same as or different than the first and/or second processing times.

Processing times needed to achieve the desired lightening of the color of the hair, e.g., desired degree or level of lift of the color of the hair, vary. Exemplary processing times may range from about 5 minutes to about 75 minutes, such as about 10 minutes to about 70 minutes, about 15 minutes to about 65 minutes, about 20 minutes to about 60 minutes, about 25 minutes to about 55 minutes, about 30 minutes to about 50 minutes, or about 30 minutes to about 40 minutes, including all ranges and subranges using any of the aforementioned as upper and lower limits. In preferred embodiments, the processing time is less than 60 minutes, such as less than 55 minutes, for example is 50 minutes, 45 minutes, 40 minutes, 35 minutes, 30 minutes, 25 minutes, 20 minutes, 15 minutes, or 10 minutes. It should be understood that for multiple-application methods of lightening hair, each processing time can be in the above ranges. Thus, for example, a first processing time may be about 50 minutes and a second processing time may be about 40 minutes, for a total processing of about 90 minutes.

Surprisingly, the amount of time needed to achieve the same or even higher levels of lift with hair lightening compositions according to the disclosure is reduced compared to traditional hair lightening compositions. For example, the processing time to achieve the same or even higher levels of lift may be reduced by about or at least 5%, about or at least 10%, about or at least 15%, about or at least 20%, about or at least 25%, or about or at least 30%. In preferred embodiments, the processing time ranges from at least 20 minutes to less than 60 minutes.

Optionally, processing may take place at room temperature or at elevated temperatures (e.g., under a hood dryer).

Thus, in various embodiments, methods according to the disclosure comprise applying a hair lightening composition to the keratin materials to be treated, e.g. hair growing from the scalp, leaving the hair lightening composition on the hair for a processing time as described above, for example ranging from about 20 minutes to about 60 minutes, or about 30 minutes to about 50 minutes, with or without the use of heat and/or foils, and subsequently removing the hair lightening composition from the hair. It is also possible to treat the hair with the hair lightening composition more than one time, e.g. two times (double processing) or more. In such embodiments, the composition will be applied to the hair, left on the hair for a desired length of time, wiped from the hair (typically without rinsing), and the hair lightening composition applied to the hair a second time and left on the hair for a desired length of time. A further wiping/reapplication can be done, or the hair can be rinsed and dried.

In additional embodiments, the methods may comprise reducing hair damage caused by lightening the hair by using hair lightening compositions according to the disclosure, compared to the damage caused by conventional hair lightening compositions.

In some embodiments, the methods comprise an additional step of mixing a hair base lightening composition with a solvent, for example at or near the time of use, and mixing the hair base mixture with an oxidizing composition or oxidizing agent at or near the time of use. It should be understood that the solvent in such embodiments could alternatively be mixed with the oxidizing agent or composition at or near the time of use. In other embodiments the methods comprise mixing an additional amount of oxidizing composition with a base lightening composition and/or oxidizing composition at or near the time of use. It should also be understood that the order of mixing is not limited, nor is the timing of mixing provided the composition is suitable for lightening the color of the hair.

### III. Kits

The disclosure further relates to kits that can be used in methods of lightening hair. The kits comprise at least two compartments or containers, such as at least three compartments or containers or more. The first compartment or container comprises a base lightening composition according to the disclosure. The second compartment or container may comprise an oxidizing composition. Alternatively, the second compartment or container may comprise an implement to mix the base composition and an oxidizing composition, and/or to apply the mixture to the hair, for example a bowl and/or a brush.

In an exemplary embodiment, the kit comprises at least three compartments or containers, where the first compartment or container comprises a base lightening composition according to the disclosure, a second compartment or container comprises an oxidizing composition, and a third compartment or container comprises an implement to mix the base composition and an oxidizing composition, and/or to apply the mixture to the hair. In such an embodiment, the hair lightening composition prepared by mixing the contents of the first and second compartments or containers would result in a hair lightening composition according to the disclosure.

In another exemplary embodiment, the kit comprises at least three compartments or containers where the first compartment or container comprises a base lightening composition according to the disclosure, a second compartment or container comprises an oxidizing composition, and a third compartment or container comprises a solvent according to the disclosure. In such an embodiment, the hair lightening composition prepared by mixing the contents of the first, second, and third compartments or containers would result in a hair lightening composition according to the disclosure.

In yet a further exemplary embodiment, the kit comprises at least three compartments or containers, where the first compartment or container comprises a base lightening composition according to the disclosure excluding one or more of the components described herein, for example excluding thermal control agents. In such an embodiment, a second compartment or container comprises the excluded component(s), for example comprises one or more thermal control agents, and a third compartment or container comprises an oxidizing composition. In such an embodiment, the hair lightening composition prepared by mixing the contents of the first, second, and third compartments or containers would result in a hair lightening composition according to the disclosure just as if the thermal control agent(s) had been included in the base lightening composition. Optionally, a fourth compartment or container which comprises an implement to mix the base composition and an oxidizing composition, and/or to apply the mixture to the hair may be included.

In still further exemplary embodiments, the kits may comprise at least one additional compartment or container comprising a solvent to be mixed with the base lightening composition. In further exemplary embodiments still, the kits may comprise at least one additional compartment or container comprising one or more additional hair care compositions, for example comprising a pre- and/or post-treatment hair conditioning composition.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “citric acid and/or salts thereof” means “citric acid and salts thereof” as well as “citric acid or salts thereof.”

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−5% from the stated number, such as +/−5%, +/−4%, +/−3%, +/−2%, or +/−1%. Unless expressly stated otherwise, “about” means+/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions in which the component is present, unless otherwise indicated.

The term “altering the color” or “color altering” as used herein refers to lifting or lightening the color of hair.

For purposes of the present disclosure, naturally-straight, natural level 4 hair that has a ΔL value of about 54 or higher when treated with a hair lightening composition as described herein is considered to achieve “high” levels of lift or lightening, naturally-straight, natural level 4 hair that has a ΔL value of about 58 or higher when treated with a hair lightening composition as described herein is considered to achieve “very high” levels of light or lightening, and naturally-straight, natural level 4 hair that has a ΔL value of about 62 or higher when treated with a hair lightening composition as described herein is considered to achieve “ultra-high” levels of lift or lightening, when measured in the CIE L* a* b* system using ColorShot MS (Newtone Technologies, software version 1.8.0).

As used herein, the terms “applying a composition onto keratin fibers” and “applying a composition onto hair” and variations of these phrases are intended to mean contacting the keratin fibers including hair, with at least one of the compositions of the disclosure, in any manner. It may also mean contacting the keratin fibers or hair in an effective amount of the composition.

As used herein, the term “salts” may include salts having a counter-ion such as an alkali metal, alkaline earth metal, or ammonium counterion. This list of counterions, however, is non-limiting. Salts also include a dissociated form of a compound, e.g. in an aqueous solution.

Unless otherwise specified for a particular component, as used herein the terms “substantially free” or “essentially free” mean the component may be added in amounts less than 2% by weight, based on the total weight of the composition in which it is present, such as less than 1.75%, less than 1.5%, less than 1.25%, less than 1%, less than 0.75%, less than 0.5%, less than 0.25%, less than 0.1%, less than 0.05%, or less than 0.01% of the specified component. Unless expressly stated otherwise, “substantially free” or “essentially free” means less than 2%. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The terms “free,” “substantially free,” and “essentially free” as used herein mean that the specific component is not added to the composition but may still be present as an additive in small amounts of a raw material that is included in the composition.

As used herein, the term “surfactants,” as well as any specifically identified surfactants, includes salts of the surfactants even if not explicitly stated.

As used herein, the term “treat” (and its grammatical variations) refers to the application of the compositions of the present disclosure onto the surface of the specified keratin material, such as hair growing from the scalp.

As used herein, the phrase “at or near the time of use” is meant to indicate that the step of mixing or combining components or compositions is performed within a short time before the mixture is applied to the keratin fibers to be treated. For example, the consumer or professional colorist may perform the mixing step within an hour or less of the mixture being applied to the hair, such as 45 minutes or less, 30 minutes or less, or 15 minutes or less.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts in each composition are expressed in percentage by weight (wt %) of active materials, unless otherwise defined, relative to the total weight of the composition.

### Evaluation Methods

In the Examples, the following procedures were used.

### Fiber Integrity Testing

Evaluation of break stress and elastic modulus was carried out using a Miniature Tensile Tester (MTT 688 from Dia-Stron Ltd) and a Fibre Dimensional Analysis System (FDAS) 770. Elastic modulus represents a measure of the hair's spring-like structure (elasticity). Elastic modulus provides fiber deformation information, so fiber with higher elastic modulus is more resistant to deformation. Break stress represents the force/area needed to break the hair fiber. A higher break stress represents a stronger hair fiber. Overall, the higher the break stress and/or the elastic modulus, the better the fiber integrity.

For each swatch, fifty (n=50) fibers per hair sample were tested at 80% relative humidity and room temperature. Each hair swatch sample was soaked in deionized (DI) water for about two (2) hours, and subsequently subjected to MTT testing. Tensile measurement was performed while the hair fibers were wet, with 2 grams of force at a traction rate of about 40 mm/min. The measured results of the hair break stress and elastic modulus were statistically analyzed using UVWin software vision 4.2.5.0, where the statistical significance level was set at p<0.05.

### Evaluation of Hair Lightening

The change in the color of hair was evaluated with the CIE L* a* b* system using Colorshot MS (Newtone Technologies, software version 1.8.0), where the change is determined by evaluating the color of the hair after treatment (L*2, a*2, b*2) compared to the color of the hair before treatment (L*1, a*1, b*1). In this system, L* indicates lightness, a* indicates red/green color or tone, and b* indicates blue/yellow color or tone. The change in lightness (ΔL) and blue/yellow tones (Δb) of the hair, which can be used to evaluate the level of lift achieved, is calculated with equations (X¹) and (X²), as follows:

$$\begin{matrix}Δ L = ( {{L}^{*}}_{2} ) - ( {{L}^{*}}_{1} ) \\ ( {X}^{1} )\end{matrix} \begin{matrix}Δ L = ( {{b}^{*}}_{2} ) - ( {{b}^{*}}_{1} ) \\ ( {X}^{2} ) .\end{matrix}$$

The change in color (ΔE, 1976) is calculated with equation (X³) as follows:

$$\begin{matrix}Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} \\ ( {X}^{3} ) .\end{matrix}$$

### Example 1—Base Lightening and Oxidizing Compositions

Comparative base lightening compositions C1-C6 and base lightening compositions 1A-1 P according to the disclosure were prepared as shown in Tables 1A-1C.

TABLE 1A
Base Lightening Compositions

| | C1 | C2 | C3 | C4 | C5 | C6 | 1A | 1B |
|---|---|---|---|---|---|---|---|---|
| UREA | 6.00 | 6.00 | 6.00 | 6.00 | 6.00 | 6.00 | 6.00 | 6.00 |
| POTASSIUM | 4.00 | 4.00 | 4.00 | 4.00 | 4.00 | 4.00 | 4.00 | 4.00 |
| CARBONATE | | | | | | | | |
| MINERAL OIL | 4.79 | 4.79 | 4.79 | 4.79 | 4.79 | 4.79 | 4.79 | 4.79 |
| SODIUM SILICATE | 28.00 | 28.00 | 28.00 | 28.00 | 28.00 | 28.00 | 28.00 | 28.00 |
| SODIUM STEARATE | 4.40 | 4.40 | 4.40 | 4.40 | 4.40 | 4.40 | 4.40 | 4.40 |
| SODIUM | 4.00 | 4.00 | 4.00 | 4.00 | 4.00 | 4.00 | 4.00 | 4.00 |
| METASILICATE | | | | | | | | |
| DISODIUM EDTA | 1.00 | 1.00 | 1.00 | 1.00 | 1.00 | 1.00 | 1.00 | 1.00 |
| HYDROXYETHYL- | 2.60 | 2.60 | 2.60 | 2.60 | 2.60 | 2.60 | 2.60 | 2.60 |
| CELLULOSE | | | | | | | | |
| ULTRAMARINES | 0.43 | 0.43 | 0.43 | 0.43 | 0.43 | 0.43 | 0.43 | 0.43 |
| (and) KAOLIN | | | | | | | | |
| AMMONIUM | 11.60 | 11.60 | 11.60 | 11.60 | 11.60 | 11.60 | 11.60 | 11.60 |
| PERSULFATE | | | | | | | | |
| ACRYLATES/C10-30 | 1.80 | 1.80 | 1.80 | 1.80 | 1.80 | 1.80 | 1.80 | 1.80 |
| ALKYL ACRYLATE | | | | | | | | |
| CROSSPOLYMER | | | | | | | | |
| SODIUM LAURYL | 1.00 | 1.00 | 1.00 | 1.00 | 1.00 | 1.00 | 1.00 | 1.00 |
| SULFATE | | | | | | | | |
| MAGNESIUM | 6.00 | 6.00 | 6.00 | 6.00 | 6.00 | 6.00 | 6.00 | 6.00 |
| CARBONATE | | | | | | | | |
| HYDROXIDE | | | | | | | | |
| POTASSIUM | 18.40 | 21.90 | 20.90 | 20.90 | 20.90 | 19.40 | 18.40 | 18.40 |
| PERSULFATE | | | | | | | | |
| GLYCINE | | 2.50 | | | 3.50 | 5.00 | 2.50 | 2.50 |
| SODIUM CITRATE | | | 3.50 | | | | 3.50 | |
| CITRIC ACID | | | | 3.50 | | | | 3.50 |
| STARCH | 6.00 | | | | | | | |

TABLE 1B
Base Lightening Compositions

| | 1C | 1D | 1E | 1F | 1G | 1H | 1I |
|---|---|---|---|---|---|---|---|
| UREA | 5.99 | 5.99 | 5.99 | 5.99 | 5.99 | 5.99 | 5.99 |
| POTASSIUM | 4.00 | 4.00 | 4.00 | 4.00 | 4.00 | 4.00 | 4.00 |
| CARBONATE | | | | | | | |
| MINERAL OIL | 4.80 | 4.80 | 4.80 | 4.80 | 4.80 | 4.80 | 4.80 |
| SODIUM SILICATE | 28.00 | 28.00 | 28.00 | 28.00 | 28.00 | 28.00 | 28.00 |
| SODIUM STEARATE | 3.93 | 3.93 | 3.93 | 3.93 | 3.93 | 3.93 | 3.93 |
| SODIUM | 4.00 | 4.00 | 4.00 | 4.00 | 4.00 | 4.00 | 4.00 |
| METASILICATE | | | | | | | |
| DISODIUM EDTA | 1.00 | 1.00 | 1.00 | 1.00 | 1.00 | 1.00 | 1.00 |
| SODIUM CITRATE | 3.50 | 3.50 | 3.50 | 3.50 | 3.50 | 3.50 | 3.50 |
| HYDROXYETHYL- | 2.60 | 2.60 | 2.60 | 2.60 | 2.60 | 2.60 | 2.60 |
| CELLULOSE | | | | | | | |
| ULTRAMARINES | 0.40 | 0.40 | 0.40 | 0.40 | 0.40 | 0.40 | 0.40 |
| (and) KAOLIN | | | | | | | |
| AMMONIUM | 11.60 | 11.60 | 11.60 | 11.60 | 11.60 | 11.60 | 11.60 |
| PERSULFATE | | | | | | | |
| ACRYLATES/C10-30 | 1.80 | 1.80 | 1.80 | 1.80 | 1.80 | 1.80 | 1.80 |
| ALKYL ACRYLATE | | | | | | | |
| CROSSPOLYMER | | | | | | | |
| SODIUM LAURYL | 1.00 | 1.00 | 1.00 | 1.00 | 1.00 | 1.00 | 1.00 |
| SULFATE | | | | | | | |
| MAGNESIUM | 5.99 | 5.99 | 5.99 | 5.99 | 5.99 | 5.99 | 5.99 |
| CARBONATE | | | | | | | |
| HYDROXIDE | | | | | | | |
| POTASSIUM | 18.90 | 18.90 | 18.90 | 18.90 | 18.90 | 18.90 | 17.90 |
| PERSULFATE | | | | | | | |
| ARGININE | 2.50 | | | | | | |
| THREONINE | | 2.50 | | | | | |
| SERINE | | | 2.50 | | | | |
| ALANINE | | | | 2.50 | | | |
| BICINE | | | | | 2.50 | | |
| TRIETHANOLAMINE | | | | | | 2.50 | |
| HYDROCHLORIDE | | | | | | | |
| GLYCINE | | | | | | | 3.50 |

TABLE 1C
Base Lightening Compositions

| | 1J | 1K | 1L | 1M | 1N | 1O | 1P |
|---|---|---|---|---|---|---|---|
| UREA | 5.99 | 5.99 | 5.99 | 5.99 | 5.99 | 5.99 | 5.99 |
| POTASSIUM | 4.00 | 4.00 | 4.00 | 4.00 | 4.00 | 4.00 | 4.00 |
| CARBONATE | | | | | | | |
| MINERAL OIL | 4.80 | 4.80 | 4.80 | 4.80 | 4.80 | 4.80 | 4.80 |
| SODIUM SILICATE | 28.00 | 28.00 | 28.00 | 28.00 | 28.00 | 28.00 | 28.00 |
| SODIUM STEARATE | 3.93 | 3.93 | 3.93 | 3.93 | 3.93 | 3.93 | 3.93 |
| SODIUM | 4.00 | 4.00 | 4.00 | 4.00 | 4.00 | 4.00 | 4.00 |
| METASILICATE | | | | | | | |
| DISODIUM EDTA | 1.00 | 1.00 | 1.00 | 1.00 | 1.00 | 1.00 | 1.00 |
| SODIUM CITRATE | 3.50 | 3.50 | 3.50 | 3.50 | 3.50 | 3.50 | 3.50 |
| HYDROXYETHYL- | 2.60 | 2.60 | 2.60 | 2.60 | 2.60 | 2.60 | 2.60 |
| CELLULOSE | | | | | | | |
| ULTRAMARINES | 0.40 | 0.40 | 0.40 | 0.40 | 0.40 | 0.40 | 0.40 |
| (and) KAOLIN | | | | | | | |
| AMMONIUM | 11.60 | 11.60 | 11.60 | 11.60 | 11.60 | 11.60 | 11.60 |
| PERSULFATE | | | | | | | |
| ACRYLATES/C10-30 | 1.80 | 1.80 | 1.80 | 1.80 | 1.80 | 1.80 | 1.80 |
| ALKYL ACRYLATE | | | | | | | |
| CROSSPOLYMER | | | | | | | |
| SODIUM LAURYL | 1.00 | 1.00 | 1.00 | 1.00 | 1.00 | 1.00 | 1.00 |
| SULFATE | | | | | | | |
| MAGNESIUM | 5.99 | 5.99 | 5.99 | 5.99 | 5.99 | 5.99 | 5.99 |
| CARBONATE | | | | | | | |
| HYDROXIDE | | | | | | | |
| POTASSIUM | 17.90 | 17.90 | 17.90 | 17.90 | 17.90 | 17.90 | 16.40 |
| PERSULFATE | | | | | | | |
| ARGININE | 3.50 | | | | | | |
| THREONINE | | 3.50 | | | | | |
| SERINE | | | 3.50 | | | | |
| ALANINE | | | | 3.50 | | | |
| BICINE | | | | | | 3.50 | |
| TRIETHANOLAMINE | | | | | 3.50 | | |
| HYDROCHLORIDE | | | | | | | |
| GLYCINE | | | | | | | 5.00 |

Oxidizing composition (developer) D1 in Table 1 D was used.

TABLE 1D
Oxidizing Composition

| | D1 |
|---|---|
| TRIDECETH-2 CARBOXAMIDE MEA | 0.85 |
| TETRASODIUM ETIDRONATE | 0.20 |
| SODIUM SALICYLATE | 0.04 |
| GLYCERIN | 0.50 |
| CETEARYL ALCOHOL | 2.28 |
| CETEARETH-25 | 0.57 |
| HYDROGEN PEROXIDE | 12.00 |
| TETRASODIUM PYROPHOSPHATE | 0.04 |
| WATER | QS |

### Example 2—Evaluation of Treated Hair

Just before use, 100 grams of base lightening compositions C1-C6 and 1A-1 P were mixed with 200 grams of developer composition D1 to produce comparative hair lightening compositions C7-C12 and hair lightening compositions 2A-2P according to the disclosure. Hair lightening composition C7, which did not include any benefit agents or carboxylic acids/salts, but included an equivalent amount of inert starch, was used as a standard.

Each of hair lightening compositions C7-C12 and 2A-2P was applied to separate swatches (swatches S1-S22) of natural level 4 hair at a rate of about 10 grams per gram of hair and covered with foil. The swatches were left to process at 33° C. for 50 minutes, rinsed, shampooed with a commercial shampoo, rinsed again, and blow dried. The change in color (ΔE), lightness (ΔL), and blue/yellow tone (Δb) of the treated hair was then evaluated using Colorshot MS, as described above (T₁). Subsequently, each swatch was treated a second time by applying the same hair lightening composition as the first at a rate of about 10 grams per gram of hair and covered with foil. The swatches were again left to process at 33° C. for 50 minutes, rinsed, shampooed with a commercial shampoo, rinsed again, and blow dried. The change in color (ΔE), lightness (ΔL), and blue/yellow tone (Δb) of the treated hair was again evaluated using Colorshot MS (T₂).

TABLE 2

| Composition | C7 | C8 | C9 | C10 | C11 | C12 | 2A | 2B | 2C | 2D | 2E |
|---|---|---|---|---|---|---|---|---|---|---|---|
| Swatch | S1 | S2 | S3 | S4 | S5 | S6 | S7 | S8 | S9 | S10 | S11 |

| Composition | 2F | 2G | 2H | 2I | 2J | 2K | 2L | 2M | 2N | 2O | 2P |
|---|---|---|---|---|---|---|---|---|---|---|---|
| Swatch | S12 | S13 | S14 | S15 | S16 | S17 | S18 | S19 | S20 | S21 | S22 |

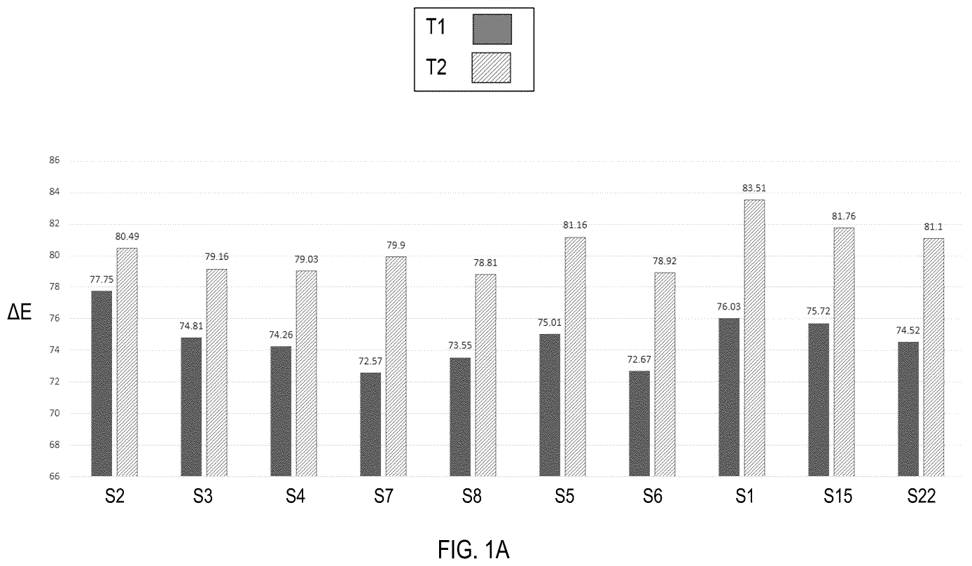

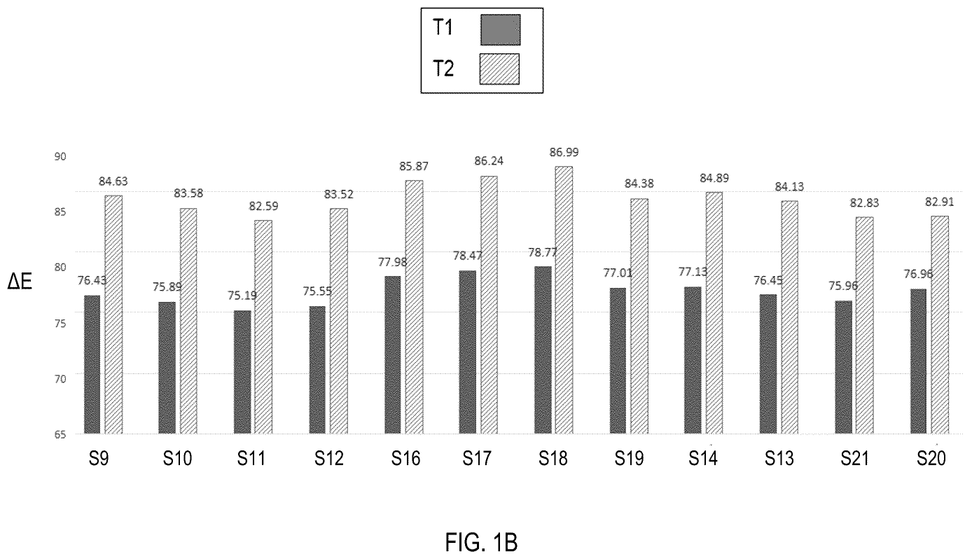

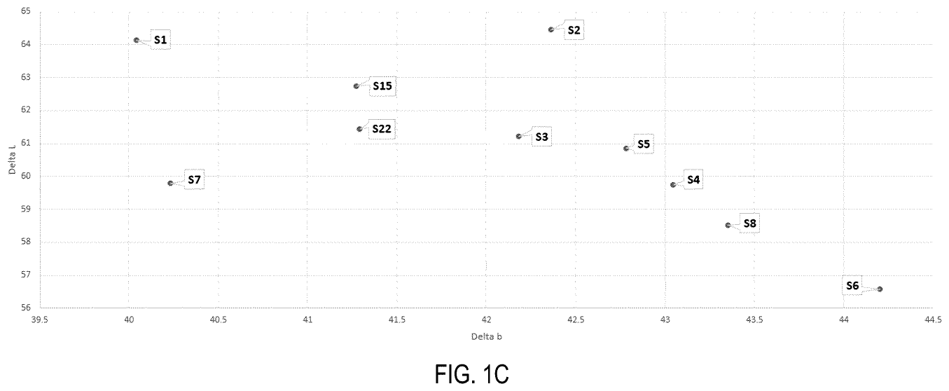

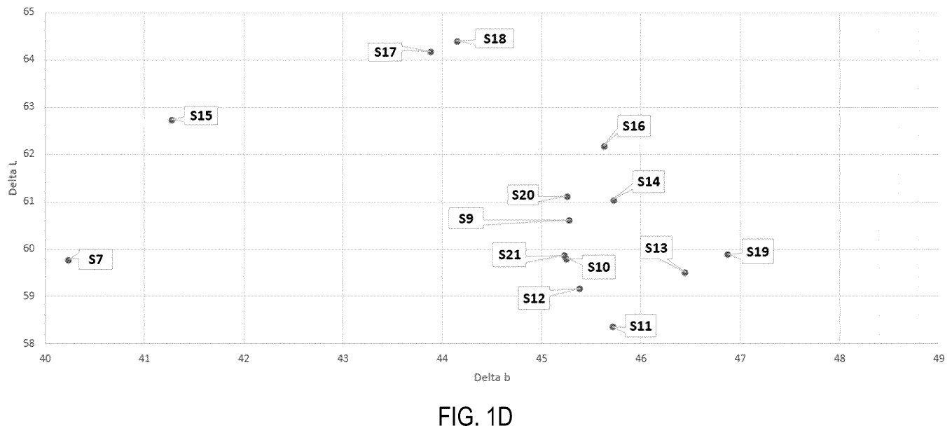

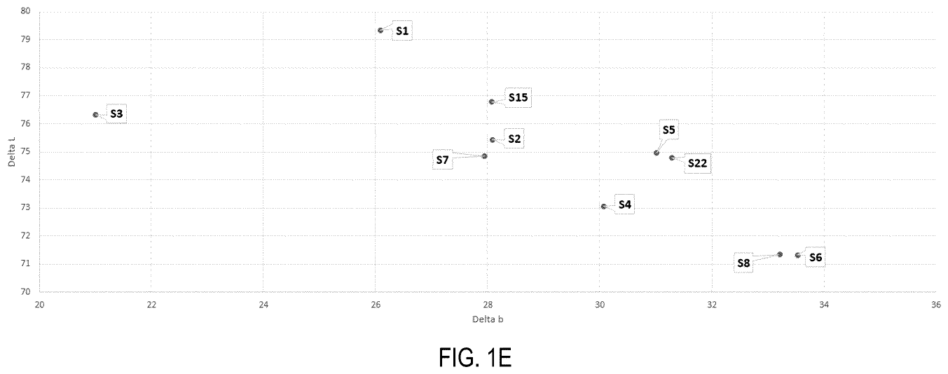

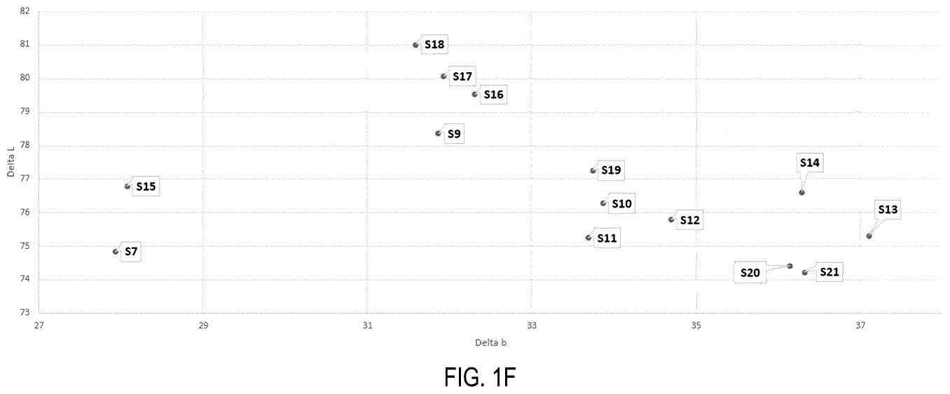

The results are shown in FIGS. 1A-1F. As seen in FIGS. 1A-1B, hair treated with hair lightening compositions having both a benefit agent and a carboxylic acid or salt had as high or higher degree of change in color (ΔE) after the second application than the hair treated with comparative lightening compositions C8-C12 which had either a benefit agent or a carboxylic acid or salt, but not both.

FIGS. 1C-1D show the difference in lightening (ΔL) and blue/yellow tone (Δb) of swatches S1-S22 at T₁. All swatches demonstrated excellent lift, with swatches S1-S2 and S15-S18 all achieving ultra-high lift.

FIGS. 1E-1F show the difference in lightening (ΔL) and blue/yellow tone (Δb) of swatches S1-S22 at T₂. All swatches demonstrated ultra-high lift. Swatch S3 had the highest level of lift of the comparative swatches. Because a difference in ΔL of less than 2 is not visible to the naked eye, all swatches treated with hair lightening compositions according to the disclosure were considered to have a degree of lift at T₂ that was equivalent to or better than that of swatch S3, other than swatch S8. However, swatch S8 was considered to have a degree of lift at T₂ that was equivalent to comparative swatches S10 and S12. Surprisingly, swatches S9 and S16-S18 demonstrated significantly increased lift with a ΔL of greater than 2 compared to swatch S3, and were noticeably lighter.

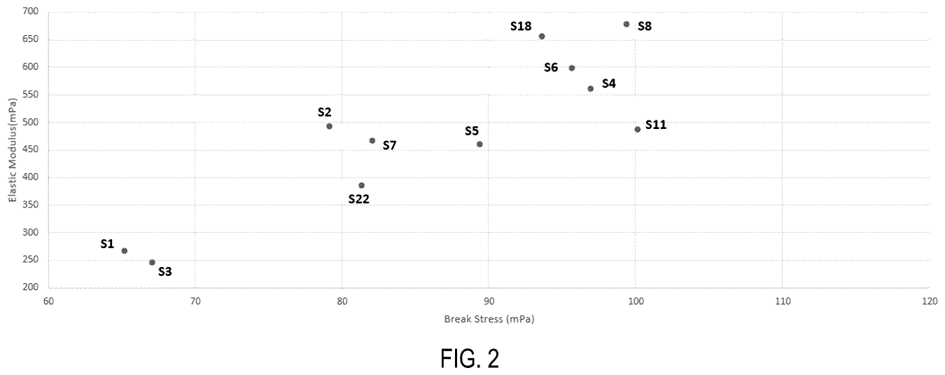

Subsequently, the break stress and elastic modulus of swatches S1-S8, S11, S18, and S22 were evaluated as described above. The results are shown in FIG. 2. As FIG. 2 shows, all of the tested swatches except S3 had better break stress and elastic modulus results than the standard (S1). However, the results for swatches S11 and S18, which also demonstrated as good or better lift results than swatch S3, show that by combining benefit agents and carboxylic acids or salts, very high or even ultra-high levels of lift can be achieved while integrity of the hair is maintained.

The results in the Examples demonstrate that base lightening compositions, base lightening mixtures, and hair lightening compositions according to the disclosure surprisingly provide very high and even ultra-high levels of hair lightening despite containing a reduced amount of persulfate compounds relative to traditional hair lightening compositions. In addition, the presence of benefit agents and carboxylic acids or salts provide significant improvement in hair fiber integrity, protecting the hair from damage during the harsh lightening process. This combination of components thus provides excellent lightening with reduced hair damage, resulting in smoother, healthier looking and feeling hair compared to hair treated with traditional hair lightening compositions.

1. A hair lightening composition made by mixing:
(a) a base lightening composition comprising:
(i) at least one persulfate compound;
(ii) at least one silicate compound;
(iii) at least one benefit agent; and
(iv) at least one carboxylic acid and/or salt thereof,
wherein the at least one benefit agent is chosen from compounds of formula (I) and salts thereof: 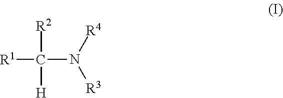
wherein:
R¹ is chosen from —C(O)OH, —CH₂C(O)OH, —(CH₂)₃N(H)C(NH₂)₂, a C1-C4 alkyl radical, or a C1-C4 hydroxyalkyl radical;
R² is chosen from hydrogen, a C1-C4 hydroxyalkyl radical, or R⁵CH₂OH;
R³ and R⁴ are independently chosen from hydrogen or a C1-C4 hydroxyalkyl radical; and
R⁵ is a C1-C4 alkyl radical;
wherein the total amount of persulfate compounds in the hair base lightening composition ranges from about 10% to not greater than 50% by weight, relative to the total weight of the base lightening composition, and
wherein the weight ratio of (a)(i):(a)(ii) is less than 1;
(b) at least one solvent,
wherein the total amount of solvent ranges from about 1% to about 25% by weight, relative to the total weight of the base lightening composition; and
(c) an oxidizing composition.

2. The hair lightening composition according to claim 1, wherein the total amount of (i) persulfate compounds in the base lightening composition ranges from about 25% to about 45% by weight, relative to the total weight of the hair base lightening composition.

3. The hair lightening composition according to claim 1, wherein the weight ratio of (a)(i):(a)(ii) in the base lightening composition ranges from about 0.1 to about 0.9.

4. The hair lightening composition according to claim 1, wherein the total amount of (b) solvent ranges from about 5% to about 20% by weight, relative to the total weight of the base lightening composition.

5. The hair lightening composition according to claim 1, wherein the weight ratio of the total amount of persulfates in the base lightening composition to the total amount of solvent ranges from about 1:1 to about 10:1.

6. The hair lightening composition according to claim 1, wherein the (iii) benefit agent is chosen from amino acids of formula (I), derivatives thereof, and/or salts thereof.

7. The hair lightening composition according to claim 1, wherein the (iii) benefit agent is present in an amount ranging from about 0.5% to about 6% by weight, relative to the total weight of the base lightening composition.

8. The hair lightening composition according to claim 1, comprising (iv) one or more salts of citric acid.

9. The hair lightening composition according to claim 1, wherein the total amount of (iv) carboxylic acids and salts thereof ranges from about 0.5% to about 6% by weight, relative to the total weight of the base lightening composition.

10. The hair lightening composition according to claim 1, wherein the weight ratio of the total amount of (iii) benefit agents to the total amount of (iv) carboxylic acids and salts thereof ranges from about 0.2 to about 2.

11. The hair lightening composition according to claim 1, prepared by mixing:
(a) a base lightening composition comprising:
(i) ammonium persulfate and at least one additional persulfate compound;
(ii) at least one silicate compound;
(iii) from about 1% to about 5% of serine; and
(iv) from about 1% to about 5% of sodium citrate,
wherein the total amount of persulfates present in the base lightening composition ranges from about 20% to about 40% by weight, relative to the total weight of the base lightening composition,
(b) water; and
(c) an oxidizing composition comprising hydrogen peroxide,
wherein the hair lightening composition is substantially free of carboxylic acids other than salts of carboxylic acids.

12. A hair base lightening composition comprising:
(i) at least one persulfate compound;
(ii) at least one silicate compound;
(iii) at least one benefit agent; and
(iv) at least one carboxylic acid and/or salt thereof,
wherein the at least one benefit agent is chosen from compounds of formula (I) and salts thereof: 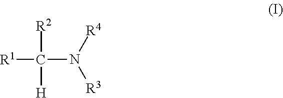
wherein:
R¹ is chosen from —C(O)OH, —CH₂C(O)OH, —(CH₂)₃N(H)C(NH₂)₂, a C1-C4 alkyl radical, or a C1-C4 hydroxyalkyl radical;
R² is chosen from hydrogen, a C1-C4 hydroxyalkyl radical, or R⁵CH₂OH;
R³ and R⁴ are independently chosen from hydrogen or a C1-C4 hydroxyalkyl radical; and
R⁵ is a C1-C4 alkyl radical; and
wherein the total amount of persulfate compounds in the hair base lightening composition ranges from about 20% to about 45% by weight, relative to the total weight of the hair base lightening composition.

13. The hair base lightening composition according to claim 12, wherein the (iii) benefit agent is chosen from amino acids of formula (I), derivatives thereof, and/or salts thereof.

14. The hair base lightening composition according to claim 12, wherein the (iii) benefit agent is present in an amount ranging from about 0.5% to about 6% by weight, relative to the total weight of the base lightening composition.

15. The hair base lightening composition according to claim 12, comprising (iv) one or more salts of citric acid.

16. The hair base lightening composition according to claim 12, wherein the total amount of (iv) carboxylic acids and salts thereof ranges from about 0.5% to about 6% by weight, relative to the total weight of the base lightening composition.

17. The hair base lightening composition according to claim 12, wherein the weight ratio of the total amount of (iii) benefit agents to the total amount of (iv) carboxylic acids and salts thereof ranges from about 0.2 to about 2.

18. The hair base lightening composition according to claim 12, comprising:
(i) ammonium persulfate and at least one additional persulfate compound;
(ii) at least one silicate compound;
(iii) from about 1% to about 5% of serine; and
(iv) from about 1% to about 5% of sodium citrate,
wherein the total amount of persulfates present in the hair base lightening composition ranges from about 20% to about 40% by weight, relative to the total weight of the base lightening composition, and
wherein the weight ratio of (iii):(iv) ranges from about 0.5 to about 1.5.

19. The hair base lightening composition according to claim 18, which is substantially free of carboxylic acids other than salts of carboxylic acids.

20. A method of lightening hair comprising:
(I) preparing a hair lightening composition by mixing:
(a) a base lightening composition comprising:
(i) at least one persulfate compound;
(ii) at least one silicate compound;
(iii) at least one benefit agent; and
(iv) at least one carboxylic acid and/or salt thereof,
wherein the at least one benefit agent is chosen from compounds of formula (I) and salts thereof: 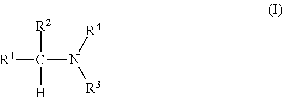
wherein:
R¹ is chosen from —C(O)OH, —CH₂C(O)OH, —(CH₂)₃N(H)C(NH₂)₂, a C1-C4 alkyl radical, or a C1-C4 hydroxyalkyl radical;
R² is chosen from hydrogen, a C1-C4 hydroxyalkyl radical, or R⁵CH₂OH;
R³ and R⁴ are independently chosen from hydrogen or a C1-C4 hydroxyalkyl radical; and
R⁵ is a C1-C4 alkyl radical;
wherein the total amount of persulfate compounds in the base lightening composition ranges from about 10% to not greater than 50% by weight, relative to the total weight of the base lightening composition, and
wherein the weight ratio of (a)(i):(a)(ii) is less than 1;
(b) at least one solvent,
wherein the total amount of solvent ranges from about 1% to about 25% by weight, relative to the total weight of the base lightening composition; and
(c) at least one oxidizing agent,
(II) applying the hair lightening composition to the hair, and
(I) removing the hair lightening composition from the hair after a desired processing time.
